(19) United States
(12) Patent Application Publication
(10) Pub. No.: US 2026/0207927 A1
(43) Pub. Date: Jul. 23, 2026

(54) MULTI-ELECTRODE LEAD WITH DIRECTIONAL ELECTRODES

(71) Applicant: SORIN CRM SAS, CLAMART (FR)

(72) Inventor: JEAN-FRANÇOIS OLLIVIER, SAINT REMY LES CHEVREUSES (FR)

(21) Appl. No.: 19/142,215

(22) PCT Filed: Dec. 22, 2022
(86) PCT No.: PCT/IB2022/000746

§ 371 Date: Jun. 22, 2025

(51) Int. Cl. A61N 1/05; A61N 1/362

(52) U.S. Cl. CPC A61N 1/056; A61N 1/362

(57) ABSTRACT

The invention relates to a multi-electrode lead device that comprises an interelectrode portion between a distal first electrode and a proximal second electrode, wherein the interelectrode portion comprises two or more additional surface electrodes (“directional” surface electrodes) having a reduced circumferential width for directional pacing and arranged in an axial sequence to obtain an elongated right bundle branch pacing area. Therefore, there is no need for multiple lead models for different anatomies and/or pre-operation examination to measure the septum wall thickness. Moreover, the pacing direction and thus the pacing efficiency of the additional directional surface electrodes can be optimized/adjusted by rotating the lead device around its longitudinal axis.

### FIELD OF THE INVENTION

The invention relates to the field of lead devices (e.g., electrode catheters) for cardiac pacing systems, such as-but not limited to-left bundle branch pacing (LBBP), cardiac resynchronization or tachycardia (“tachy”) systems.

### BACKGROUND OF THE INVENTION

Different electrical activation sequences of cardiac pacing may lead to different mechanical pump efficiencies of a stimulated heart. What is needed is a fast and homogenous contraction of heart ventricles to optimize pump efficiency.

Traditional pacing sites such as the right ventricular apex (RVA) may provide a stable lead position with low displacement rate but are not very effective to optimize left ventricle (LV) contraction (representing about 80% of the heart mass). Long-term RVA pacing may have deleterious effects on left ventricular function by inducing an iatrogenic left bundle branch block (LBBB), which can have strong influences on the left ventricle hemodynamic performances. This observation led to a reassessment of traditional approaches and to a research of alternative pacing sites in order to get to more physiological pattern of ventricular activation and to avoid deleterious effects.

LBBP has emerged as an alternative method for delivering physiological pacing to achieve electrical synchrony of the LV, especially in patients with infranodal atrioventricular block and/or LBBB. Proximal LBBs run through the LV septum and fan out to form a wider target for pacing compared to the His bundle. A technique for LBBP has been developed using a ventricular transseptal approach (i.e., pacing the LV from the RV). LBBP has been reported to offer low pacing thresholds and large R waves, and because the distal conduction system is targeted, has a lower theoretical risk for development of distal conduction block.

After an initial site for an LBBP location at the right surface of the ventricular septum has been determined, the pacing lead (i.e., a helical fixation element or electrode at the lead tip) is screwed into the LV septum, e.g., by puncturing the tissue with the distal tip of the helical fixation element (fixation helix). The LBBP lead depth into the LV septum may be determined by at least one of observing changes in the notch in V1 lead, sheath angiography, fulcrum sign, and impedance monitoring. The pacing lead is slowly progressed into the determined depth (e.g., approximately 6 to 8 mm) by the application of a torque, meanwhile avoiding any perforation of the septum. Finally, LBB capture is confirmed based on acceptable pacing parameters. The confirmation may be based on at least one of a paced morphology of an RBBB pattern, a recording of an LBB potential, a stimulus-peak of the LVAT that shortens abruptly with increasing output or remains shortest and constant at low and high outputs, a selective LBBP and a non-selective LBBP, and a recording of a retrograde His potential or anterograde LBB potential during pacing.

Common features of implantation or placement processes include transvenous access, transseptal placement of the pacing lead into the LV septal subendocardium in the LBB region, and confirmation of capture of the LBB.

However, variations in patient's anatomy may account for certain differences in interventricular thickness of the LV septum, which may vary from about 10 mm to about 20 mm. In addition to uncertain overall septum thickness, the location of the LBB conductive fibers deep inside the septal tissue is also variable between patients, typically located closer to the left ventricle border of the septum, such as about 1-5 mm therefrom. To assure successful left ventricle capture at the lowest capture threshold, the distal electrode of the lead may need to be deployed as close to the LBB fibers as possible, while avoiding the risk of penetrating through the septum and entering the left ventricular cavity.

Moreover, in cases of a combined LBB and RBB pacing, control of both LBB and RBB needs to be achieved by respective correctly positioned LV and RV cathodes. The delivered electrical pulses need to be designed for low energy consumption. From a geometric aspect, this means that at least a section of the RV cathode needs to be as close as possible to the RBB fibers and at least a section of the LV cathode needs to be as close as possible to the LBB fibers.

### SUMMARY OF THE INVENTION

It is an object of the present invention to provide a lead device that is configured to be usable for patients or device recipients despite differences in the anatomy of their septum (especially its thickness) (“various anatomies” hereinafter), while ensuring low energy consumption.

This object is achieved by a lead device as claimed in claim 1.

The proposed lead device comprises an interelectrode portion between the distal first electrode and the proximal second electrode, wherein the interelectrode portion comprises two or more additional surface electrodes (“directional” surface electrodes) having a reduced circumferential area (or width) for directional pacing and arranged in an axial sequence to obtain an elongated (right bundle branch) pacing area.

In bipolar leads with first and second electrodes (e.g., an anode and at least one cathode), one electric channel conducts the electrical pacing pulse towards the lead tip and a distal first electrode (e.g., cathode) and the other channel completes the circuit back to the pacemaker via the proximal second electrode (e.g., anode). Myocardial capture requires a minimum local current density (i.e., ratio between pacing current (electrons) and pacing area) at the contact area (exposed area) of the first electrode (cathode). A smaller exposed area leads to an increased current density and also an increased resistance, which preserves battery life as the current flow is reduced.

It is noted that the present invention may as well be used in connection with a unipolar lead. In this case, the second electrode (e.g., anode) may be provided at or may correspond to a housing of a pacing device (e.g., an implantable pacemaker) and the interelectrode portion may range from the first electrode to the pacing device. In this case, a connector (e.g., an IS1 connector) with a single connection electrode or terminal can be used at the proximal end of the lead device.

Accordingly, the additional two or more directional surface electrodes with reduced circumferential width provide a reduced exposed area for low energy/battery consumption by achieving an increased current density and also provide an elongated joint longitudinal electrode length for an enhanced pacing area. From a geometric aspect this means that, e.g., for various anatomies of the septum, at least a section of one of the two or more directional surface electrodes can be placed close to the RBB fibers, while at least a section of the LV electrode is placed close to the LBB fibers. Therefore, there is no need for multiple lead models for different anatomies and/or pre-operation examination to measure the septum wall thickness. Moreover, the limited pacing direction and thus the pacing efficiency of the directional pacing electrode closest to the LBB fibers can be optimized/adjusted by rotating the lead device around its longitudinal axis.

The substrate or body of the elongated surface electrode (i.e., a combination of pacing surface and insulated surface) can be configured as a single physical component to maintain a lead design with desired change of stiffness (stiffness gradient) in an area to be inserted into the septum. A change in stiffness leads to a change in flexion of the lead tip and an increased risk of fracture. A plurality of pacing surfaces can thus be arranged (e.g., embedded) on the elongated surface electrode, which may all be electrically connected together and connected to one unique terminal or electrode of a connector at the proximal end of the lead device. As it can be structured as a single mechanical element, it can be configured to provide a continuous stiffness of a desired amount that protects the lead tip. If there were many separate electrodes, this would create a (sudden) stiffness gradient at each separate electrode, which might harm the lead tip during the puncturing process, and this is avoided. Also thereby, the total pacing surface of the elongated surface electrode can be spread over a longer axial distance to maintain good electrical performance by at least one of increasing the probability of having some portion of the elongated surface electrode as close as possible to the tissue area to be stimulated (e.g., the RBB) for various anatomies (e.g., thicknesses of the septum) and maintaining a desired cumulated reduced pacing surface (e.g., 2 to 8 mm²). The pacing surface can be continuous or discontinuous along the axial and/or circumferential length of the elongated surface electrode, which may thus consist of one or more separated pacing surface areas that may be electrically connected together.

According to a first option, the additional surface electrodes may be connected to only one (i.e., a single) pacing input terminal of a proximal lead connector. Thereby, a simple and robust structure with a standard connector (e.g., IS4 connector) can be used for connecting the additional surface electrodes.

According to a second option that can be combined with the above first option, the additional surface electrodes may be formed by a single conductive element that is partially covered by an electrically insulator coverage. Thereby, the structure and manufacturing of the lead device can be simplified and made robust.

According to a third option that can be combined with any one of the above first and second options, the additional surface electrodes may comprise a first additional surface electrode and a second additional surface electrode both having an angular width of substantially a half circumference and located on opposite areas of the circumference of the interelectrode portion. Thereby, a simple and robust structure with opposite pacing directions can be achieved.

According to a fourth option that can be combined with any one of the above first and second options, wherein the additional surface electrodes may comprise three or four additional surface electrodes having an angular width of less than a quarter circumference. Thereby, three or four pacing directions can be achieved to increase flexibility and efficiency of the pacing effect while keeping energy/battery consumption low.

According to a fifth option which can be combined with any one of the first to fourth options, the distal first electrode may be formed by a fixation helix. Thereby, the first electrode can be used to screw the lead tip into the tissue of the septum.

According to a sixth option which can be combined with any one of the first to fifth options, the proximal second electrode may be an anode. Thereby, all electrodes can be provided at the lead tip and can be easily connected to a proximal connector (e.g., a standard IS4 connector).

According to a seventh option which may be combined with the fifth or sixth option, a ratio between a first outer diameter of the fixation helix and a second outer diameter at a distal end of the interelectrode portion may be set between 0.8 and 1, the first outer diameter may be set between 1 and 1.8 mm, the length of the elongated surface electrode may be set between 7 and 11 mm, and the length of the fixation helix may be set between 1.5 and 5 mm. These dimensions facilitate the screwing process of the lead tip into the tissue.

According to an eighth option which can be combined with any one of the first to seventh options, the interelectrode portion may have a conical shape. The conical shape reduces the resistance of the lead tip when entering the tissue during the screwing process.

According to a ninth option which can be combined with any one of the first to eighth options, the body of the lead device may have a coradial structure. The coradial structure enables a lead design that is less bulky and has less stiffness.

According to a tenth option which can be combined with any one of the first to ninth options, the axial distance between the distal end of the fixation helix and the distal end of the additional surface electrodes my range from 7 to 12 mm, the cumulated axial length of the additional surface electrodes may range from 7 to 11 mm, and the axial distance between the distal end of the additional surface electrodes and the distal end of the proximal second electrode may range from 10 to 20 mm.

According to an eleventh option which can be combined with any one of the first to tenth options, the additional surface electrodes may be configured to obtain a cumulated size of a resultant limited active electrode surface within a range of 2 to 8 mm². Thereby, good electrical performance can be achieved, while preserving current consumption and thus device longevity.

It shall be further understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically a heart with a lead device placed for ventricular transseptal LBB pacing;

FIG. 2 shows schematically a side view of a lead device according to a first embodiment with a first cathode formed by a fixation helix and an elongated second cathode arrangement formed by two directional surface electrodes with limited exposure;

FIG. 3 shows schematically a cross sectional front view of the elongated second cathode structure of the first embodiment;

FIG. 4 shows schematically a side view of a lead device according to a second embodiment with a first cathode formed by a fixation helix and an elongated second cathode arrangement formed by three directional surface electrodes with limited exposure; and

FIG. 5 shows schematically a cross sectional front view of the elongated second cathode structure of the second embodiment.

### DETAILED DESCRIPTION OF EMBODIMENTS

Various embodiments of the present invention are now described based on an improved lead device (e.g., electrode catheter) with fixation helix. Although the present invention is particularly advantageous within the context of transseptal pacing such as LBBP, the invention is not limited thereto and may also be used in connection with other pacing types and/or sites for other applications that require placement of a lead device within a body tissue.

It is noted that throughout the present disclosure only those elements, portions, components and/or devices that are relevant for the proposed lead device and placement operation are shown in the accompanying drawings. Other elements, portions, components and/or devices have been omitted for reasons of brevity. Furthermore, components designated by same reference signs or numbers are intended to have the same or at least a similar function, so that their function is not described again later.

Furthermore, throughout the present disclosure, “proximal” and “distal” are terms that are used to indicate distances from an operating end (reference point) of the lead device, where the physician or other user controls the screwing process. Proximal is closer to the operating end, while distal is further away (at a greater distance) from the operating end.

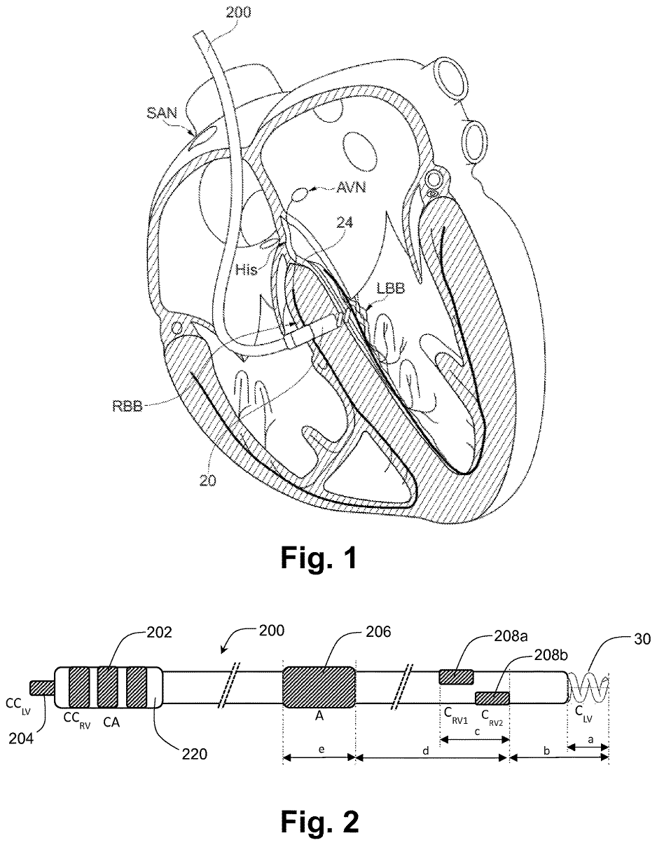

FIG. 1 shows schematically a heart with an inserted lead device 200, where the pacing lead tip 20 is placed for ventricular transseptal LBBP. Thereby, the LV can be paced from the RV by a ventricular transeptal approach as a guide for catheter delivery. The placement of the pacing lead tip 20 may be performed based on the procedure explained above. LBBP may be defined as capture of the LBB (i.e., left bundle trunk or its proximal fascicles/fibers), usually with septal myocardium capture at low output (e.g., <1.0 V/0.4 ms).

In a normal cardiac function, the heartbeat starts in the heart itself due to the sinoatrial node (SAN) which is found in the top of the right atrium (RA) and sets the rate at which the heart contracts. It sends out electrical impulses that are carried through the muscular walls of both atria. These impulses cause atrial systole. The impulse is then passed to another node within the heart-the atrioventricular node (AVN). This node is in the lower part of the RA. Once the impulse from the SAN reaches the AVN the impulse is passed to conducting fibers which travel down the central wall of the heart. The impulse then splits and travels up the LV and RV causing them to contract simultaneously (ventricular systole).

Important elements of the conduction system of the heart are found within the septum 24. The His bundle travels in the subendocardium down the right side of the septum 24 for about 1 cm before dividing into the LBB and RBB. The RBB continues down the right side of the septum 24, while the LBB crosses to the left side and splits into anterior and posterior divisions.

Under normal circumstances, excitation from the SAN controls the heart rhythm. An abnormality in the sinus rhythm leads to arrhythmia, which refers to abnormalities in the rate, rhythm, site of origin, and conduction of the cardiac electrical pulse. When disorders occur in specific intraventricular conduction fibers, the repolarization wave must then travel through the slower muscle-muscle conduction to reach the ventricles. Classic disorders related to conditions that involve different conduction bundle branches include LBBB and RBBB. An electrocardiogram (ECG), as obtained from an inserted lead device, can be used to measure and record cardiac electrical activities and thus can provide important information on cardiac functions. The ECG has been used as a standard diagnostic tool to analyze arrhythmia.

In embodiments, the lead tips of pacing or tachy leads are designed to avoid a risk of perforation of the septum. They may also be equipped with a soft tip (made of e.g. silicone) to increase a stop surface. That is, when the helical fixation element or electrode (called “helix” hereinafter) is engaged with (e.g., screwed into) the (cardiac) issue, this tissue is pushed against the soft tip to stop the helix from rotation and further progression within the tissue. The length of the helix may be limited, e.g., to an active length of about 2 mm. The lead and/or the helix need to be designed for optimizing required energy/force for puncturing and for allowing a well-controlled and safe progression without increasing complexity of the lead.

Furthermore, according to embodiments, the body of the lead device may be configured to improve slipperiness of a contact with a guiding catheter used for guiding the lead device (e.g., through a blood vessel) to a target area. This can be achieved by using e.g. a polyurethane (PU) material with reduced diameter to allow an advancement progress of the lead body through the guiding catheter and the lead tip 20 through the septum 24 with limited effort.

Suitable designs of lead devices according to embodiments may have a multi-lumen, coaxial and coradial structure, both as tachycardia leads or bradycardia leads, and a central lumen for a stylet passage may be provided. Coaxial leads have an inner conductor that extends down the length of the lead to the tip electrode (helix), the cathode, arranged in a coil configuration that provides a central lumen e.g. to allow for passage of a stylet at implantation.

Coradial bipolar leads address some of the disadvantages of coaxial leads with respect to the bulk and stiffness of their four-layer design, by providing a new conductor and insulator technology where a single coil extends down the length of the lead (again with a central lumen to allow for stylet insertion) and consists of two parallel, alternating conductor strands, one of which connects to the cathode and the other to the anode. Each conductor strand may be individually coated with a bonded layer of e.g. ethylene tetrafluoroethylene (ETFE) fluoropolymer insulation that serves to insulate each strand from the other, despite being intertwined. The single, two-component coil may be surrounded by a single, outer insulation covering.

The multi-lumen or coaxial or coradial leads may optionally comprise a fixed, non-retractable helix to minimize size. However, a retractable helix may also be used in connection with the described embodiments.

Furthermore, the proposed multi-electrode lead device according to embodiments may be configured to provide improved torquability, i.e., an ability to transmit torque safely and accurately to the helix (e.g., full lead body torque) and stylet-driven compatibility to ease the handling (e.g., by push transmission). In an example, a coradial lead with compatible screwing stylet (screwdriver stylet) may be provided.

The following embodiments of the proposed multi-electrode lead device are configured to be useable for multiple various anatomies with different thicknesses of the septum and/or structures of the RBB and/or LBB fibers and compatible with applicable standards (e.g., the international standard IS4 which is usable for a maximum of four independent electrical lines). This is achieved by providing in the interelectrode portion between the distal first electrode (e.g., helix cathode) and the proximal second electrode (e.g., surface anode) an additional elongated surface electrode arrangement with two or more surface electrodes (directional surface electrodes) with reduced circumferential length to provide a directional pacing characteristic in a correspondingly limited angular section around the lead tip 20. The reduced circumferential length of the directional surface electrodes leads to a reduced exposed pacing surface of the elongated second electrode arrangement for increased current density (i.e., less pacing area for the pacing current). The increased current density allows to reduce the amount of power supplied to the additional elongated surface electrode arrangement and thereby reduce energy/battery consumption. More specifically, in addition to the first cathode formed by the helix, a second cathode can be formed by the additional longitudinally elongated electrode arrangement with reduced exposed surface for increased current density. The embodiments are directed to different options of designing the directional surface electrodes of the additional elongated electrode arrangement, e.g., by changing at least one of the number, size and angular coverage of the directional surface electrodes.

The elongated second cathode arrangement with the one or more directional surface electrodes allows to provide two more flexible pacing sites or sides (e.g., for concurrent LBB and RBB pacing) for various anatomies. Thereby, the multi-electrode lead can be adapted e.g. to various septum wall thicknesses. To reach an effective pacing threshold, the total exposed conductive surface of the elongated cathode arrangement needs to physically contact tissue and expose a limited pacing surface.

The two pacing electrodes (i.e., the first cathode and the additional elongated second cathode arrangement) may be electrically independent by using at least one of different timings, different thresholds, different impedances and the like.

FIG. 2 shows schematically a side view of a multi-electrode lead device 200 with a lead tip according to a first embodiment with a first cathode (C_{LV}) formed by a fixation helix 30 and a second cathode arrangement (C_{RV1}, C_{RV2}) formed by two separate surface electrodes 208a and 208b having a limited circumferential width and being arranged in a longitudinal (axial) sequence (i.e., longitudinally (axially) neighboring positions) to provide an elongated longitudinal pacing area.

The lead tip of the lead device 200 comprises the fixation helix 30 with an active length a that is screwed into heart tissue (septum tissue) by puncturing the tissue with the distal tip of the fixation helix 30. The lead device 200 can be used for stimulation of the LBB and RBB. The lead device 200 may for example include an elongate body that extends between a proximal end with a connector 220 that is configured to interface with an implantable pulse generator and a distal end at the fixation helix 30. The elongate body may also include a lumen that extends between the proximal end and the distal end.

In at least some of the following embodiments, as regards the design of the distal end (distality) of the lead device 200, the rate/ratio between the outer diameter of the helix 30 and the outer diameter of the housing of the lead tip may be larger than 70% ideally 100%, wherein an isoprofil distality may be provided to avoid a front stop surface for better insertion.

Furthermore, the helix 30 may be made of a rigid material to avoid deformation of the helix 30 during screwing, while a fixed helix 30 (i.e., a lock between the helix 30 and the lead body) may simplify handling (i.e., no parasite tool is required for a retractable system).

Moreover, design flexibility may be provided by adapting a distance b between the fixation helix 30 and the proximal elongated second cathode arrangement for both-side pacing and/or the longitudinal length c of the elongated second cathode arrangement to be suitable for a desired range of thicknesses of the septum in different people.

The distal design of the lead device 200 may further be configured to allow smooth and predictable advancement of the lead tip into the septum until the helix (cathode) 30 reaches the desired location at the LV chamber, i.e., close to the LBB without full perforation of the septum, so that the helix 30 does not protrude into the LV chamber.

Additionally, the design of the lead device 200 may be configured to minimize the required energy/torque to perform the puncturing of the septum. This may be achieved by providing a dedicated distally tapered lead tip (not shown) with a conical shape of an interelectrode portion between a proximal end of the helix 30 and a distal end of a proximal anode (A) 206 with an axial or longitudinal length e. The distal end of the anode 206 is located at a longitudinal distance d from the distal end of the elongated second cathode.

In certain instances, at least a proximal portion of the fixation helix 30 may be insulated and at least one turn of the distal end of the fixation helix 30 may be uninsulated. One or more turns of the fixation helix 30 (e.g., internal to the lumen of the elongate body) may be covered by a dielectric or other insulative material. The proximal portion or turn of the fixation helix 30 being eliminated may minimize impedance interference that can result from the spacing of a proximal electrode (not shown) and the fixation helix 30.

The fixation helix 30 may be mounted (e.g., welded) on a driver (not shown) which may comprise a surrounding coil or other non-flat regular or irregular surface structure (not shown) to ensure a good adhesion of the surrounding material of the lead body to the driver between proximal end of the fixation helix 30 and the distal end of the anode 206 to thereby obtain a simple rigid and durable structure of the lead tip with low number of components for improved long-term reliability. The driver (not shown) may be fixedly supported in the lead body and mechanically and electrically connected to a screwing stylet (not shown) adapter matched for insertion of a coupling end (engagement portion) of a separate screwing stylet with a screwdriver function for allowing rotational driving of the fixation helix 30 via the driver. Due to an electrical connection between the fixation helix 30 and the screwing stylet, electrical signals sensed by the fixation helix 30 at the target area can be routed via the screwing stylet to a signal analyzer and used for monitoring correct placement of the fixation helix 30 during the screwing operation without any disconnection, allowing a single step operation.

The conical shape of the lead tip may for example be based on dimensional parameters of an outer diameter Da of the proximal anode 206, an outer diameter Dl of the distal section of the lead tip, an outer diameter Dh of the fixation helix 30, the length a of the fixation helix 30, and total length Lt (e.g., e+d+b) of the lead tip including the fixation helix 30 and a tapered portion of the lead body (e.g., surrounding the driver) between the fixation helix 30 and the proximal anode 206.

In examples, the rate/ratio Dh/Dl can be set between 0.8 and 1, while Dh can be set between 1 and 1.55 mm (preferably 1.40 mm). Da can be set between 1.25 mm and 1.94 mm (preferably 1.66 mm), the length a of the fixation helix 30 can be set between 2 and 5 mm. The difference b−a can be set between 5 and 7 mm, the longitudinal length c of the elongated second cathode can be set between 7 and 11 mm, the distance d can be set between 10 and 20 mm, and the longitudinal length e of the anode 206 can be set between 5 and 10 mm.

The proposed specific conical shape with the above-mentioned ranges of dimension ensures that the lead tip with the fixation helix 30 can be used to puncture tissue in the target area in a controlled and smooth manner providing a conic profile minimizing the required energy to perform the puncture.

In the specific example shown in FIG. 2 and subsequent FIG. 4, the connector 220 at the proximal end of the lead device 200 comprises four independent connector electrodes (terminals) including three circumferential connector electrodes 202 and one axial connector electrode 204. This connector design corresponds to the standard IS4 connector and is configured to provide connections for a maximum of four electrical lines or wires of the lead device 200. In the specific example, three of the four connector electrodes are used for connecting to the anode 206, the elongated second cathode arrangement 208a, 208b and the first cathode at the fixation helix 30. The axial connector electrode 204 corresponds to a first connector electrode (CC_{LV}) 204 that connects via a first line to the fixation helix 30 (cathode C_{LV} for LV pacing). A first one (CC_{RV}) of the circumferential connector electrodes 202 connects via a second line to both surface electrodes 208a (C_{RV1}) and 208b (C_{RV2}) of the elongated second cathode arrangement (for RV pacing). Finally, a second one (CA) of the circumferential connector electrodes 202 connects via a third line to the anode (A).

Alternatively, the non-used fourth one of the four connector electrodes may be used for connecting one of the two surface electrodes 208a, 208b of the elongated second cathode arrangement, so that the two surface electrodes 208a, 208b are connected to different connector electrodes and can thus be driven by different pacing signals to provide an enhanced pacing control function.

The anode 206 can be used as a sensing electrode with a large surface (e.g., 40 mm²) and may be structures as a single electrode or two electrodes.

In the embodiment of FIG. 2, the elongated second cathode arrangement is formed by two separate directional surface electrodes 208a, 208b with different pacing directions, that may be connected via a common single inner (internal) connection to a wire or line that connects to the most proximal one of the three circumferential connector electrodes 202 of the connector 220. They may for example be formed by a single mechanical element (conductive electrode) with a cylindrical shape that is partially coated by an insulating coverage (e.g., a parylene coating or other electrically insulating coating) to provide the two separated exposed electrode surfaces. Alternatively, the single mechanical element may comprise the two conductive surface elements and a connecting portion that connects the two elements, wherein only the connection portion is covered by the insulating coverage. Thereby, a simple and robust design can be achieved.

Thus, the elongated second cathode arrangement comprises a first exposed area (first directional surface electrode 208a) shaped as a ring segment at a proximal portion (e.g., proximal half) of the elongated second cathode arrangement and a second exposed area (second directional surface electrode 208b) shaped as a ring segment at the distal portion (e.g., distal half) of the elongated second cathode arrangement. In an example, the total exposed area of the two exposed ring segment areas may sum up to a value from 4 mm² to 8 mm².

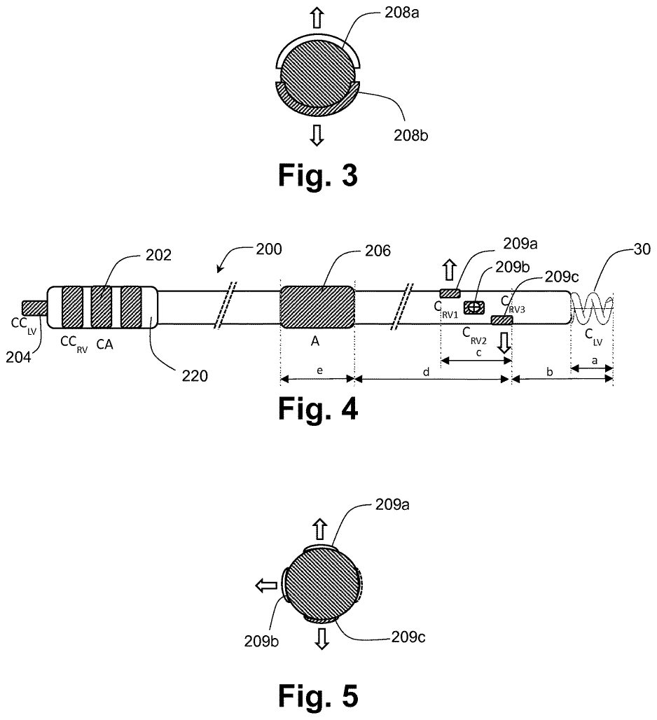

FIG. 3 shows a cross sectional front view of the elongated electrode structure of the first embodiment, wherein the radial cutting plane crosses the second directional surface electrode 208b.

As can be gathered from FIG. 3, the angular width of the ring segments of the first and second directional surface electrodes 208a, 208b covers substantially 180° in opposite directions of the lead tip. As indicated by the arrows in FIG. 3, the upper surface electrode 208a directs the pacing effect in the upward direction of FIG. 3, while the lower surface electrode 208b directs the pacing effect in the downward direction of FIG. 3. The two exposed ring segment electrode areas (cathodes) 208a and 208b may be connected to the same inner wire to simplify the internal design and provide a robust electrode structure. The reduced exposed area increases the current density during pacing and thereby allows reduced energy/battery consumption.

FIG. 4 shows schematically a side view of a multi-electrode lead device according to a second embodiment with a first cathode (C_{LV}) formed by a fixation helix 30 and an elongated second cathode arrangement having a longitudinal length c and formed by three separate directional surface electrodes 209a to 209c with different pacing directions, that may be connected via a common single inner connection to a wire or line that connects to the most proximal one of the three circumferential connector electrodes 202 of the connector 220. The first connector electrode (CC_{LV}) 204 connects via a first line to the fixation helix 30 (cathode C_{LV} for LV pacing). A first one (CC_{RV}) of the circumferential connector electrodes 202 connects via a second line to the three directional surface electrodes 209a (C_{RV1}), 209b (C_{RV2}) and 208c (C_{RV3}) of the elongated second cathode arrangement (for RV pacing). Finally, a second one (CA) of the circumferential connector electrodes 202 connects via a third line to the anode (A).

Alternatively, the non-used fourth one of the four connector electrodes may be used for connecting one of the three surface electrodes 209a to 209c of the elongated second cathode arrangement, so that one of the three directional surface electrodes 209a to 209c is connected to different connector electrode and can thus be driven by different pacing signals to provide an enhanced pacing control function. As another alternative, the proximal connector 220 may comprise a fifth connector electrode (not shown) that may be used for connecting an additional one of the three surface electrodes 209a to 209c of the elongated second cathode arrangement, so that each of the three directional surface electrodes 209a to 209c is connected to a different connector electrode and can thus be driven by an own dedicated pacing signal to provide a further enhanced pacing control function.

The three directional surface electrodes 209a to 209c may for example be formed by a single mechanical element (conductive electrode) with a cylindrical shape that is partially coated by an insulating coverage (e.g., a parylene coating or other electrically insulating coating) to provide the three separated exposed electrode surfaces. Alternatively, the single mechanical element may comprise the three conductive surface elements and connecting portions each connecting respective two elements of the three conductive surface elements, wherein only the connection portions are covered by the insulating coverage. Thereby, a simple and robust design can be achieved.

Thus, the elongated second cathode arrangement comprises a first exposed area (first directional surface electrode 209a) shaped as a ring segment at a proximal portion (e.g., proximal third) of the elongated second cathode arrangement, a second exposed area (second directional surface electrode 209b) shaped as a ring segment at a central portion (e.g., central third) of the elongated second cathode arrangement, and a third exposed area (third directional surface electrode 209c) shaped as a ring segment at a distal portion (e.g., distal third) of the elongated second cathode arrangement. In an example, the total exposed area of the three exposed ring segment areas may sum up to a value from 4 mm² to 8 mm².

It is noted that, contrary to the example of FIG. 4, the three directional surface electrodes 209a to 209c may be arranged at equal or non-equal angular distances around the circumference of the lead tip.

FIG. 5 shows a cross sectional front view of the elongated electrode structure of the first embodiment, wherein the radial cutting plane crosses the third directional surface electrode 209c.

As can be gathered from FIG. 5, the angular width of each of the ring segments of the first to third directional surface electrodes 209a to 209c covers less than 90° in different directions of the lead tip. As indicated by the arrows in FIGS. 4 and 5, the first directional surface electrode 209a directs the pacing effect in the upward direction of FIGS. 4 and 5, the second directional surface electrode 209b directs the pacing effect in a sideward direction of FIGS. 4 and 5, and the third directional surface electrode 209c directs the pacing effect in the downward direction of FIGS. 4 and 5. The three exposed ring segment electrode areas (cathodes) 209a to 209c may be connected to the same inner wire to simplify the internal design and provide a robust electrode structure. The reduced exposed area increases the current density during pacing and thereby allows reduced energy/battery consumption.

As indicated by a dotted shape in FIG. 5, a fourth directional surface electrode may be provided opposite to the second directional surface electrode 209b to direct the pacing effect to the other (opposite) sideward direction of FIGS. 4 and 5 and thereby provide an equal distribution of directional surface electrodes around the circumference of the lead tip.

Throughout the embodiments described above, the directional surface electrodes 208a, 208b, 209a to 209c may be ring segments with rectangular shape and may be arranged at substantially equal distances around the circumference of the lead tip. As an alternative, other shapes (oval, circular, triangular etc.) may be provided and/or more than four neighboring (sequential) directional surface electrodes may be provided in the axial direction. The electrode patterns of the directional surface electrodes may or may not overlap each other in the axial and/or in the circumferential direction of the lead tip. Thereby, a continuously or stepwise varying current densities can be achieved along the axial and/or circumferential direction of the lead tip. This varying current density can be used for controlling pacing efficiency during the puncturing process via the helix.

Thereby, the total exposed area of the elongated second cathode arrangement can be reduced, while all directional surface electrodes of the elongated second cathode arrangement may be connected to a single inner wire or line to simplify the internal design and provide a robust electrode structure. The reduced exposed area increases the current density during pacing and thereby allows reduced energy/battery consumption. Moreover, the limited angular coverage of the directional surface electrodes provides a directional pacing effect that can be controlled by a rotational movement of the lead tip to enhance the pacing efficiency by directing pacing to desired tissue regions.

As an example which is applicable to all above embodiments, the directional surface electrodes may be configured so that a limited size of the active electrode surface remains, which ranges between 2 and 8 mm².

The electrode pattern(s) of the directional surface electrodes can be produced by a selective insulation coverage (e.g., parylene coating which provides high insulation and good biocompatibility) achieved by e.g. a mechanical masking of the surface of the limited pacing surface that shall remain and/or by (numerically controlled) selective laser ablation of a full coated electrode surface (locally removing the insulation coverage) and/or by laser texturing to add surface material.

To summarize, multi-electrode lead devices that comprise an interelectrode portion between a distal first electrode and a proximal second electrode have been described, wherein the interelectrode portion comprises two or more additional surface electrodes having a reduced circumferential width for directed pacing and arranged in an axial sequence to obtain an elongated pacing area (e.g., RBB pacing area). Therefore, there is no need for multiple lead models for different anatomies and/or pre-operation examination to measure tissue dimensions (e.g., septum wall thickness). Moreover, the pacing direction and thus the pacing efficiency of the directional surface electrodes can be optimized/adjusted by rotating the lead device around its longitudinal axis.

While the invention has been illustrated and described in detail in the drawings and foregoing description, such illustration and description are to be considered illustrative or exemplary and not restrictive. The invention is not limited to the disclosed embodiments. It can be applied to various types of lead devices (e.g., bradycardia or tachycardia lead devices with multi-lumen, coaxial or coradial structure) and applications in the field of cardiac pacing or sensing systems.

The proposed multi-electrode lead device 200 with elongated electrode arrangement may be configured to be adapted or adaptable to IS4 (low voltage) or DF4 (high voltage) connectors or other connector types. It can be used in connection with leadless pacemakers that would have the elongated second cathode arrangement in contact with the RV septal branch and the first cathode (helix) able to go through the septum so has to reach the LBB and deliver a synchronized pacing pulses on both ventricles. Thereby, no additional hardware is required.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. The foregoing description details certain embodiments of the invention. It will be appreciated, however, that no matter how detailed the foregoing appears in the text, the invention may be practiced in many ways, and is therefore not limited to the embodiments disclosed. It should be noted that the use of particular terminology when describing certain features or aspects of the invention should not be taken to imply that the terminology is being re-defined herein to be restricted to include any specific characteristics of the features or aspects of the invention with which that terminology is associated.

1. A lead device (200) for left and/or right bundle branch pacing, the lead device (200) comprising:
a distal first electrode (30) configured to be inserted into a septum of a heart by puncturing and to be used for left bundle branch pacing;
a proximal second electrode (206); and
an interelectrode portion between the distal first electrode (30) and the proximal second electrode (206);
wherein the interelectrode portion comprises two or more additional surface electrodes (208a, 208b; 209a-209c) having a reduced circumferential area for directional pacing and arranged in an axial sequence to obtain an elongated pacing area.

2. The lead device (200) of claim 1, wherein the additional surface electrodes (208a, 208b; 209a-209c) are connected to only one pacing input terminal of a proximal lead connector (220).

3. The lead device (200) of claim 1 or 2, wherein the additional surface electrodes (208a, 208b; 209a-209c) are formed by a single conductive element that is partially covered by an insulating coverage.

4. The lead device (200) of any one of the preceding claims, wherein the additional surface electrodes (208a, 208b) comprise a first additional surface electrode (208a) and a second additional surface electrode (208b) both having an angular width of substantially a half circumference and located on opposite areas of the circumference of the interelectrode portion.

5. The lead device (200) of any one of claims 1 to 3, wherein the additional surface electrodes comprise three or four additional surface electrodes (209a to 209c) having an angular width of less than a quarter circumference.

6. The lead device (200) of any one of the preceding claims, wherein the distal first electrode is formed by a fixation helix (30).

7. The lead device (200) of any one of the preceding claims, wherein the proximal second electrode is an anode (206).

8. The lead device (200) of claim 6, wherein a ratio between a first outer diameter of the fixation helix (30) and a second outer diameter at a distal end of the interelectrode portion is set between 0.8 and 1, the first outer diameter is set between 1 and 1.8 mm, the length of the elongated surface electrode (208a-c; 209; 212) is set between 7 and 11 mm, and the length of the fixation helix (30) is set between 1.5 and 5 mm.

9. The lead device (200) of any of the preceding claims, wherein the interelectrode portion has a conical shape.

10. The lead device (200) of any of the preceding claims, wherein the body of the lead device (200) has a coradial structure.

11. The lead device (200) of any of the preceding claims, wherein the axial distance between the distal end of the fixation helix (30) and the distal end of the additional surface electrodes (208a, 208b; 209a-209c) ranges from 7 to 12 mm, the cumulated axial length of the additional surface electrodes (208a, 208b; 209a-209c) ranges from 7 to 11 mm, and the axial distance between the distal end of the additional surface electrodes (208a, 208b; 209a-209c) and the distal end of the proximal second electrode (206) ranges from 10 to 20 mm.

12. The lead device (200) of any of the preceding claims, wherein the additional surface electrodes (208a, 208b; 209a-209c) are configured to obtain a cumulated size of a resultant limited active electrode surface within a range of 2 to 8 mm².
